(19) United States
(12) Patent Application Publication
(10) Pub. No.: US 2002/0061281 A1
(43) Pub. Date: May 23, 2002

(54) Aerosolized anti-infectives, anti-inflammatories, and decongestants for the treatment of sinusitis

(76) Inventors: Robert S. Osbakken, Camarillo, CA (US); Mary Anne Hale, Woodland Hills, CA (US); Frederick T. Leivo, Carpinteria, CA (US); James D. Munk, Camarillo, CA (US)

Correspondence Address: MORGAN, LEWIS & BOCKIUS, 1800 M STREET NW, WASHINGTON, DC, 20036-5869, US

(21) Appl. No.: 09/942,959

(22) Filed: Aug. 31, 2001

Related U.S. Application Data
(63) Continuation-in-part of application No. PCT/US00/18410, filed on Jul. 5, 2000.
(63) Continuation-in-part of application No. 09/577,623, filed on May 25, 2000.
(60) Provisional application No. 60/142,618, filed on Jul. 6, 1999.
(60) Provisional application No. 60/142,620, filed on Jul. 6, 1999.
(60) Provisional application No. 60/142,621, filed on Jul. 6, 1999.
(60) Provisional application No. 60/142,622, filed on Jul. 6, 1999.
(60) Provisional application No. 60/142,624, filed on Jul. 6, 1999.
(60) Provisional application No. 60/142,741, filed on Jul. 6, 1999.
(60) Provisional application No. 60/142,881, filed on Jul. 6, 1999.
(60) Provisional application No. 60/193,507, filed on Apr. 3, 2000.
(60) Provisional application No. 60/193,508, filed on Apr. 3, 2000.
(60) Provisional application No. 60/193,509, filed on Apr. 3, 2000.
(60) Provisional application No. 60/193,510, filed on Apr. 3, 2000.
(60) Provisional application No. 60/194,078, filed on Apr. 3, 2000.

(51) Int. Cl. A61L 9/04; A61K 9/00

(52) U.S. Cl. 424/43

(57) ABSTRACT

Pharmaceutical compositions are described that comprise one or more active ingredients selected from the group consisting of an anti-infective agent, anti-inflammatory agent, anti-mucolytic agent, antihistamine, an antiseptic, and antibiotic combinations or combinations of others of these classes of ingredients, and particularly to compositions formulated as a solution or suspension in a unit dose for aerosol administration to treat chronic sinusitis.

### RELATED APPLICATIONS

This application is a Continuation-In-Part of PCT/US00/18410, filed Jul. 5, 2000 and U.S. application Ser. No. 09/577,623, filed May 25, 2000, both of which claim the benefit of U.S. Provisional Applications 60/142,618, 60/142,620, 60/142,621, 60/142,622, 60/142,624, 60/142,741, and 60/142,881, all filed on Jul. 6, 1999, and of U.S. Provisional Applications 60/193,507, 60/193,508, 60/193,509, 60/193,510, and 60/194,078, all filed on Apr. 3, 2000. All of the above-listed applications are hereby incorporated by reference in their entireties.

### TECHNICAL FIELD

The present invention relates to pharmaceutical compositions comprising one or more active ingredients selected from the group consisting of anti-infective agents, anti-inflammatory agents, mucolytic agents, antihistamines, antileukotrienes, decongestants, anticholinergics and antiseptics and particularly to compositions formulated into a liquid, for example, as a solution, suspension, or emulsion, in a unit dose or multi-dose vials for aerosol administration to treat chronic sinusitis.

### BACKGROUND OF THE INVENTION

There are a number of air-filled cavities called sinuses in the skull (Stedman's Medical Dictionary, 27th Edition, page 1644, (1999), Lippincott Williams & Wilkins, Baltimore, Md.). Four pairs of sinuses known as the paranasal sinuses, connect the space (known as the nasal passage) running from the nostrils and up through the nose. These four pairs of paranasal sinuses are the frontal sinuses, the maxillary sinuses, the ethmoid sinuses, and the sphenoid sinuses. They are located, respectively, in the forehead, behind the cheekbones, between the eyes, and behind the eyes. A membrane lining the sinuses secretes mucus, which drains into the nasal passage from a small channel in each sinus. Healthy sinuses are sterile and contain no bacteria. In contrast, the nasal passage normally contains many bacteria that enter through the nostrils as a person breathes.

A number of factors and/or processes are involved in maintaining healthy sinuses. The mucus secreted by the membrane lining must be fluid but sticky, in order to flow freely yet absorb pollutants and entrap bacteria. It must also contain sufficient amounts of bacteria-fighting substances, such as antibodies. Additionally, small hair-like projections called cilia, located in the nostril, must beat in unison to propel mucus outward, in order to expel bacteria and other particles. Moreover, the mucous membranes themselves must be intact, and the sinus passages must be open to allow drainage and the circulation of air through the nasal passage. When one or more of these processes or factors are amiss, causing obstruction of the sinus passage, an infection called sinusitis develops.

Sinusitis is an inflammation of the membrane lining one or more paranasal sinuses. There are three different types of sinusitis: acute, recurrent acute, and chronic. As an example, acute bacterial sinusitis is characterized as lasting less than three weeks or occurring less than four times a year and can be successfully treated using antibiotics, leaving no damage to the linings of the sinus tissue. Recurrent acute sinusitis occurs more often but leaves no significant damage. Chronic sinusitis lasts longer than three weeks and often continues for months. In cases of chronic sinusitis, there is usually tissue damage. According to the Center for Disease Control (CDC), thirty seven million cases of chronic sinusitis are reported annually.

Causes of Sinusitis

The most common cause for sinusitis is a viral cold or flu that infects the upper respiratory tract and causes obstruction. Obstruction creates an environment that is hospitable for bacteria, the primary cause of acute sinusitis (Etkins et al., 1999 Nidus Information Services, Inc. Well-Connected Report: Sinusitis. June 1999. (Online) www.well-connected.com). The bacteria most commonly found in acute sinusitis are Streptococcus pneumoniae (also called pneumococcal pneumonia or pneumococci), H. influenzae (a common bacteria associated with many respiratory infections in young children), and Moraxella (or Branhamella) catarrhalis. Less common bacterial culprits include Pseudomonas and other streptococcal strains including Staphylococcus aureus.

Fungi are an uncommon cause of sinusitis, but its incidence is increasing. The fungus Aspergillus is the common cause of fungal sinusitis. Others include Curvularia, Bipolaris, Exserohilum, and Mucormycosis. Fungal infections can be very serious and should be suspected in people with sinusitis who also have diabetes, leukemia, AIDS, or other conditions that impair the immune systems. Fungal infections can also occur in patients with healthy immune systems. There have been a few reports of fungal sinusitis caused by Metarrhizium anisopliae which is used in biological insect control.

Chronic or recurrent acute sinusitis can be a lifelong condition and may result from untreated acute sinusitis that causes damage to the mucous membranes, medical disorders that cause chronic thickened stagnant mucus, or abnormalities in the nasal passage such as polyps, enlarged adenoids, cleft palate, or tumors. The same organisms that cause acute sinusitis are often present in chronic sinusitis. In addition, about 20% of chronic sinusitis cases (Etkins et al., 1999, Id.) are caused by Staphylococcus aureus (commonly called Staph infection). Along with these bacteria, certain anaerobic bacteria, particularly the species Peptostreptococcus, Fusobacterium, and Prevotella, are found in 88% of cultures in chronic sinusitis cases (Etkins et al., 1999, Id.). Fungi can also cause chronic and recurrent sinusitis. An uncommon form of chronic and highly recurrent sinusitis is caused by an allergic reaction to fungi, usually, aspergillus, growing in the sinus cavities. Fungal sinusitis usually occurs in younger people with healthy immune systems and is more likely to be found in warm climates.

Symptoms of Sinusitis

In acute sinusitis, symptoms almost always present are nasal congestion and discharge which is typically thick and contains pus that is yellowish to yellow-green. Severe headache occurs, and there is pain in the face. A persistent cough occurs particularly during the day. Other upper respiratory symptoms and fever may be present. Sneezing, sore throat, muscle aches, and fatigue are rarely caused by sinusitis itself, but may result from symptoms or causes, such as muscle aches caused by fever, sore throat caused by post-nasal drip, and sneezing resulting from allergies.

The symptoms of recurrent acute and chronic sinusitis tend to be vague and generalized, last longer than eight weeks, and occur throughout the year, even during nonallergy seasons. Nasal congestion and obstruction are common. Yellowish discharge, chronic cough, bad breath, and postnasal drip may occur. Sufferers do not usually experience facial pain unless the infection is in the frontal sinuses, which results in a dull, constant ache. However, facial tenderness or pressure may be present.

Site-specific symptoms depend on the location of the infection. Frontal sinusitis causes pain across the lower forehead. Maxillary sinusitis causes pain over the cheeks and may travel to the teeth, and the hard palate in the mouth sometimes becomes swollen. Ethmoid sinusitis causes pain behind the eyes and sometimes redness and tenderness in the area across the top of the nose. Sphenoid sinusitis rarely occurs by itself. When it does, the pain may be experienced behind the eyes, across the forehead, or in the face. Rare complications of sinusitis can produce additional symptoms which may be severe or even life threatening.

Treatments of Sinusitis

The primary objectives for treatment of sinusitis are reduction of swelling, eradication of infection, draining of the sinuses, and ensuring that the sinuses remain open. Less than half of patients reporting symptoms of sinusitis need aggressive treatment and can be cured using home remedies and decongestants alone. Steam inhalation and warm compresses applied over the sinus are often sufficient to relieve discomfort. Many over-the-counter decongestants are available, either in tablet form or as sprays, drops, or vapors, which bring the medication into direct contact with nasal tissue.

Antibiotics are prescribed if decongestants fail to relieve symptoms or if other problems exist, including signs of infection (such as yellowish nasal discharge). They prevent complications, relieve symptoms, and reduce the risk of chronic sinusitis. Most patients with sinusitis caused by bacteria can be successfully treated with antibiotics used along with a nasal or oral decongestant.

Chronic sinusitis is often difficult to treat successfully, however, as some symptoms persist even after prolonged courses of antibiotics. The usefulness of antibiotics in treating chronic sinusitis is debated. Steroid nasal sprays are commonly used to treat inflammation in chronic sinusitis. For patients with severe chronic sinusitis, a doctor may prescribe steroids, such as prednisone. Since oral steroids can have serious side effects, they are prescribed only when other medications have not been effective.

When medical treatment fails, surgery may be the only alternative in treating chronic sinusitis. Studies suggest that most patients who undergo surgery have fewer symptoms and better life. Presently, the most common surgery done is functional endoscopic sinus surgery, in which the diseased and thickened tissues from the sinuses are removed to allow drainage. This type of surgery is less invasive than conventional sinus surgery, and serious complications are rare.

Considerations and Concerns of Treatments

Sprays, drops, and vapors work quickly but often require frequent administration. Nasal decongestants may dry out the affected areas and damage tissues. With prolonged use, nasal decongestants become ineffective. The tendency is to then increase the frequency of use to as often as once an hour. Withdrawal from the drugs after three to five days of over-frequent use can itself cause symptoms of sinusitis and the return of nasal congestion phenomenon known as rebound effect. Short-acting nasal decongestants may cause rebound effect after only eight hours. Rebound effect leads to dependency when the patient takes the decongestant to treat the rebound effect, the drug becomes ineffective, the patient withdraws, and the condition rebounds again, with the nasal passages becoming swollen and burning. Eventually, the condition can become worse than before the medication was taken. Nasal decongestants are generally recommended for no more than one to three days of use because of this risk.

Some oral decongestants may cause constriction of other vessels in the body, temporarily raising blood pressure in people with hypertension. Other side effects of oral decongestants include insomnia, agitation, abnormal heart rhythms (particularly in people with existing cardiac problems), and urinary retention in men with enlarged prostates. Decongestant sprays and drops, too, are absorbed into the body and can sometimes cause these side effects.

The most common side effect for nearly all antibiotics is gastrointestinal distress. Antibiotics also double the risk for vaginal infections in women. Certain drugs, including some over-the-counter medications, interact with antibiotics, and all antibiotics carry the risk for allergic reactions, which can be serious in some cases. Thus, patients should inform their physician of all medications they are taking and of any drug allergies.

Oral antibiotics are usually prescribed for 7 to 10 days. Patients must take all of the tablets prescribed; failure to do so may increase the risk for reinfection and also for development of antibiotic-resistant bacteria. It should be noted, however, that even after antibiotic treatments, between 10% and 25% of patients still complain of symptoms.

Of major concern to physicians and the public is the emergence of bacterial strains that have become resistant to common antibiotics due to frequent exposure. It should be noted that the average person is not yet endangered by this problem. The risk is greatest in hospitals and nursing homes, but it is still not high. Nonetheless, the prevalence of such antibiotic-resistant bacteria has increased dramatically worldwide, and caution should be exercised.

Nebulization Therapy

Nebulization is a conventional treatment for pulmonary infections related to cystic fibrosis, because it is relatively easy and safe to use, and because it delivers antibiotics topically to the site of infection, with little systemic absorption of the antibiotics. Nebulization has also been known to have been used for sinus infections and pulmonary infections, related to bronchiectasis. Thus, there are few systemic side effects.

### Small Aerosolized Particles for Treating Sinusitis

Yokota et al., Japanese Journal of Antibiotics, 609(15):48 (1995), reports administration of cefmenoxime using a nebulizer to treat sinusitis patients. These authors evaluated cefmenoxime against clinical isolates from sinusitis patients, and found that minimum inhibitory concentrations were lower when a one percent (1%) solution was used with a nebulizer. The paper speculates that sufficient concentrations exceeding such minimum inhibitory concentrations would be obtained by nebulizer treatment using a cefmenoxime nasal solution.

Guevaraetal., Anales O. R. L. Iber.-Amer. XVIII, 3:231-238 (1991), describes aerosol therapy for treating patients suffering from chronic sinusitis. The disclosed aerosol therapy involves delivery of a therapeutic composition comprising 500 mg of cefotaxime, 5 mg metylprednisolone, and 1.5 ml N-acetylcystine using an air-jet nebulizer for 15-20 minutes, every 8 hours, over a total period of 15 days. The air-jet nebulizer produces aerodynamic particle diameters of average mass of four microns. Guevara et al. reports a success rate of 96%. However, Guevara et al. does not disclose adding a surfactant to assist deposition, penetration, and retention of the antibiotic in the sinuses.

Kondo et al., Acta Otolaryngol. Suppl., 525: 64-67 (1996), reports treatment of paranasal sinusitis using fosfomycin (FOM) aerosol. Kondo et al. describes delivery of 4 ml of 3% FOM solution using either a jet-type nebulizer or an ultrasonic nebulizer. The jet-type nebulizer produces aerosol particles having about 0.5 to 0.7 μm in diameter, while the ultrasonic-type nebulizer produces particles having about 2-4 μm in diameter. The results of Kondo et al. indicate that the ultrasonic-type nebulizer delivers a higher concentration of FOM to the maxillary sinus surface and is therefore more effective in treating paranasal sinusitis than the jet-type nebulizer. Although Kondo et al. suggests that the preferred aerosol particle size is about 2-4 μm in diameter for deposition of a higher level of antibiotic in the maxillary sinus, Kondo et al. does not disclose an administration schedule or the addition of a surfactant to the FOM solution to further increase the deposition of FOM in the sinuses.

### Small Aerosolized Particles for Pulmonary Treatment

Smith et al., U.S. Pat. No. 5,508,269, discloses the use of aminoglycoside aerosol formulations to treat patients suffering from endobronchial infection. Smith et al. describes delivery of the aminoglycoside formulation using a jet or ultrasonic nebulizer that produces aerosol particle size between 1 and 5 μm. The formulation comprises 200 to 400 mg of aminoglycoside dissolved in about 5 ml of solution containing 0.225% sodium chloride, and it has a pH between 5.5 to 6.5. Although Smith teaches delivery of aminoglycoside to the endobronchial space using a nebulizer for the treatment of endobronchial infection, Smith does not teach an aerosol formulation for treatment of sinusitis and does not disclose a treatment schedule. It is also noted that the aerosol particle size disclosed in Smith et al. is a broad range. It is not predictable what fraction of the aerosol particles between 1 to 5 μm will deposit in the sinuses, and what fraction of the aerosol particles will have a diameter of 1 μm, 2 μm, etc.

Rubin et al., U.S. Pat. No. 5,925,334, describes the use of aerosolized surfactant to promote pulmonary airway clearance. The method of Rubin et al. comprises administering a formulation containing a surfactant using a PARI LC Jet nebulizer for 15 minutes, 3 times a day for 14 consecutive days, to patients suffering from bronchitis or cystic fibrosis. However, Rubin does not teach the use of aerosolized antibiotic or aerosolized antibiotic and surfactant combination to treat sinusitis.

Schmitt et al., U.S. Pat. No. 4,950,477, teaches a method of preventing and treating pulmonary infection by fungi using aerosolized polyenes. The method comprises administering to a patient suffering from pulmonary infection by aspergillus about 0.01 mg/kg to 6.0 mg/kg of a polyene in an aerosol of particles having an aerodynamic diameter between about 0.5 μm to about 8 μm. Schmitt et al. specifically discloses the administration of amphotericin B. Although Schmitt et al. teaches aerosolized polyenes for treatment of pulmonary infection, Schmitt et al. does not provide guidance for using aerosolized polyenes for treating sinusitis.

O'Riordan et al., Journal of Aerosol Medicine, 20(1):13-23 (1997), reports the effect of nebulizer configuration on delivery of aerosolized tobramycin to the lung. O'Riordan et al. discloses the delivery of tobramycin using either an ultrasonic nebulizer delivering aerosol particles having between 1.45 to 4.3 μm or a jet nebulizer delivering aerosol particles having about 1.25 μm. The results of O'Riordan et al. show that nebulizer configuration affects both the amount of aerosolized tobramycin inhaled as well as the particle size. Specifically, nebulizers that produce large particles are prone to considerable deposition on tubing and connections. O'Riordan et al. recommends that nebulizer configuration be specified in treatment protocols.

Large Particle Aerosolization

In contrast to the references discussed above, Negley et al., ENT Journal, 78(8):550-554 (1999), and Desrosiers et al., (presented at the ENT Academy Meeting, May 1999) teach large particle nebulization therapy for treatment of sinusitis. Negley observes that deposition of medication into the sinuses is best achieved when the aerosolized particles are 16 to 25 μm in size. Desrosiers et al. reports that large particle saline aerosol therapy alone is effective in treating refractory sinusitis and that the addition of tobramycin to the saline solution had minimal benefit.

The journal articles and patents discussed above teach various aerosol therapies for the treatment of sinusitis. However, there does not appear to be agreement among the various authors as to the optimal size or size distribution of the aerosolized particles or even whether antibiotics are effective in treating sinusitis. What has been needed is a clinically effective anti-infective treatment protocol for sinusitis, a more optimal therapy schedule, and an appropriate nebulizer configuration for the deposition of aerosolized anti-infective particles into the sinuses for the successful and consistent treatment of chronic sinusitis.

Antileukotrienes

Leukotrienes play a key role in inflammatory responses and are involved in generating many different inflammatory pathologies. Leukotrienes are produced and released from inflammatory cells, including eosinophils and mast cells. The release of leukotrienes from inflammatory cells induces bronchoconstriction, mucous secretion, and increased vascular permeability (Dahlen et al., Nature, 288:484-486 (1980); Smith et al., Am Rev Respir Dis, 131:368-372 (1985); Adelroth et al., N Engl J Med., 315:480-484 (1986)).

Leukotrienes are derived from a common precursor, leukotriene A4 (LTA4). The latter is formed only after an intermediate step in which hydroxyperoxyeicosatrienoic acid (5-HPETE) is synthesized by the action of 5-lipoxygenase (5-LO) on arachidonic acid (AA). Thus, the use of antileukotrienes to block the 5-LO route is one possible way of inhibiting the production of the leukotrienes involved in the inflammatory processes (Bell et al., Journal of Lipid Mediators, 6:259-264 (1993); R. M. McMillan et al., Trends Pharmacy. Sci., 13:323-330 (1992)). An alternative way to inhibit leukotrienes is the use of antileukotrienes that are leukotriene receptor antagonists.

Antileukotrienes that block leukotrienes at the receptor level have been shown to be relatively safe and effective in the treatment of chronic mild to moderate asthma. Montelukast sodium (Singulair®) is an example of such an antileukotriene. It is a potent, oral, specific leukotriene D4-receptor agonist (cysteinyl leukotriene [CysLT1]-receptor antagonist) and has recently been approved for the treatment of chronic asthma in patients aged 6 years and older (Reiss et al., Arch Intern Med., 158: 1213-1220 (1998); Reiss et al., Am J Respir Crit Care Med., 155:A662 (1997); Reiss et al., Am J Respir Crit Care Med., 151:A378 (1995); Reiss et al., Eur Respir J., 19(suppl):289S (1995)).

Lane, S. J. (Respiratory Medicine, 92:795 (1998)) reviews leukotriene antagonism in asthma and rhinosinusitis. According to Lane, leukotrienes have been shown to be involved in the pathogenesis of bronchial asthma and to contribute to the inflammation of allergic rhinitis. Moreover, inhibition of leukotrienes has been shown to be associated with an improvement in these disease states. Lane proposes that agents active in the 5-LO pathway such as zileuton (5-lipoxygenase inhibitor), zafirlukast, montelukast, and pranlukast (all three are inhibitors of the leukotrienes at the receptor level) are likely to be alternatives for treating both asthma and rhinosinusitis as the efficacy of these drugs is established. However, Lane does not teach aerosolized leukotriene compositions for treating sinusitis.

Antihistamines

In contrast to leukotrienes, histamine (His) is not an inflammation mediator, but is involved in the physiological alteration during the established inflammatory processes. Histamine is stored in mastocytes and basophils and is released by these cells in response to certain stimuli which effect dilation of the blood vessels. This dilation is accompanied by a lowering of blood pressure and an increased permeability of the vessel walls, so that fluids escape into the surrounding tissues. This reaction may result in a general depletion of vascular fluids, causing a condition known as histamine poisoning or histamine shock. Allergic reactions in which histamine is released, resulting in the swelling of body tissue, show similarities to histamine poisoning. The release of histamine might also be partly responsible for difficult breathing during an asthma attack.

In the 1930s the Italian pharmacologist Daniel Bovet (1907-1992) working in Paris, discovered that certain chemicals counteracted the effects of histamine in guinea pigs. However, the first antihistamines were too toxic for use on humans. By 1942, they had been modified for use in the treatment of allergies.

More than 25 antihistamine drugs are now available (“Histamine,” Microsoft® Encarta® Online Encyclopedia 2000 http://encarta.msn.com® 1997-2000 Microsoft Corporation. All rights reserved.). They are categorized into the following classes:

1. Ethanolamines: diphenhydramine hydrochloride, dimenhydrinate, carbinoxamine, clemastine fumarate, bromodiphenhydramine hydrochloride.

2. Ethylenediamines: tripelennamine hydrochloride, pyrilamine maleate, antazoline phosphate, methapyriline.

3. Alkylamines: chlorpheniramine maleate, brompheniramine maleate, dexchlorpheniramine maleate, dimethindene maleate, triprolidine hydrochloride, pheniramine maleate.

4. Piperzines: cyclizine hydrochloride or lactate, meclizine hydrochloride, hydroxyzine hydrochloride, hydroxyzine pamoate, buclizine, chlorcyclizine.

5. Phenothiazines: promethazine hydrochloride, methdilazine, trimeprazine tartrate.

6. Miscellaneous: cyproheptadine, ketotifen, azatadine maleate, terfenadine, fexofenadine, astemizole.

Antihistamines do not cure, but help relieve nasal allergy symptoms such as: congestion, itching, and discharge; eye symptoms such as: itching, burning, tearing, clear discharge; skin conditions such as: hives, eczema, itching and some rashes; and other allergic conditions. Antihistamines may relieve symptoms of allergy accompanying a cold, or they may have an anticholinergic effect that dries cold secretions, but they do not have any influence on viral infections, which are the cause of colds (“Antihistamine,” Microsoft® Encarta® Online Encyclopedia 2000 http://encarta.msn.com® 1997-2000 Microsoft Corporation. All rights reserved.).

Pharmaceutical compositions of antihistamines for therapeutic use are well-known to the skilled artisan. Wenig et al., U.S. Pat. No. 4,749,700, discloses compositions comprising antihistamine, antinausea, and antiemetic agents for nasal administration via liquid sprays or drops to a patient in need thereof Nasal delivery provides enhanced bioavailability, minimized variations in blood levels, and more rapid onset of activity and reduced dosages as compared to administration such as oral, subcutaneous, intramuscular, or by way of suppository. Although Wenig et al. discusses the use of antihistamine to treat various conditions including sinusitis, Wenig et al. does not describe effective particle size for nasal sprays or the inclusion of a surfactant for delivery.

Gordziel et al., U.S. Pat. No.6,037,358, discloses tannate compositions which are antihistaminic for the symptomatic relief of coryza associated with common cold, sinusitis, allergic rhinitis, and upper respiratory tract conditions. However, Gordziel et al. does not teach aerosolization of the tannate compositions for nasal delivery. Nor does Gordziel et al. teach specific formulations comprising a surfactant and size of aerosolized particles for effective delivery to the sinuses.

Histamine type 1 (H1)-receptor antagonists have been used extensively in the treatment of allergic diseases such as rhinitis. Loratidine (Claritin®) is a selective H1-receptor antagonist devoid of significant sedative or anticholinergic properties. In vitro, loratidine inhibits leukotriene C4 synthesis. In vivo, it has been shown to inhibit histamine release and to decrease eosinophil counts in blood and sputum (Reicin et al., Arch Intern Med 160:2481 (2000)).

Braun et al. (Allergy, 52(6): 650 (1997), discloses that H1-blockers are routinely added to the standard treatment of acute sinusitis and describes studies using loratidine to treat acute sinusitis. Braun et al. reports that patients receiving loratidine were significantly improved compared to patients receiving placebo and that loratidine in addition to standard therapy improved the control of some symptoms of sinusitis. Although the prior art teaches treatment of sinusitis using loratidine, Braun et al. does not provide aerosolized loratidine of specific particle size for delivery to patients suffering from sinusitis.

Antiseptics

Examples of antiseptics include, but are not limited to iodine, chlorhexidine acetate, sodium hypochlorite, and calcium hydroxide.

Topically, iodine has been used as an antiseptic to inhibit infection. Iodine is a broad spectrum antimicrobial agent that has bactericidal, fungicidal and viricidal properties.

U.S. Pat. No. 4,355,021 discloses a substantially dry, impregnated wipe having iodine and a means for retaining the iodine. The iodine is present in the wipe in an amount from about 1% to about 15% by weight of the wipe and in an amount sufficient to provide viricidal activity. Iodine is preferably present in an amount of from about 2% to about 5% in a facial tissue.

U.S. Pat. No. 5,897,872 discloses a nasal moisturizing solution containing iodine. The iodine-containing nasal moisturizer solution is useful for the prevention and/or treatment of sinusitis, sino-nasal congestion, acute or chronic rhinosinusitis, viral nasopharyngitis, allergic rhinitis, inhalant allergy, and related conditions associated with nasal congestion. The iodine-containing nasal moisturizing saline solution may be applied to the mucous membranes of the nose by using nose drops or a nose spray. Although the patent discloses treatment of sinusitis by delivering the nasal moisturizing solution containing iodine via nose spray, the patent does not teach adjusting the surface tension of the solution to, for example between 10 to 70 dynes/cm. Moreover, the patent does not teach aerosolized particles having a mass median aerodynamic diameter in the range of about 1.0 to 4.0 microns.

Waltimo et al., Int Endod J, 32:421(1999), describes the use of iodine potassium iodide to kill Candida albicans in vitro. Candida albicans is a fungal organism known to produce sinusitis. Waltimo et al., reports that iodine potassium iodide is more effective than calcium hydroxide against Candida albicans. However, the reference does not teach treatment of patients diagnosed with sinusitis using iodine potassium iodide.

Antibiotic Combinations

Emergence of bacterial resistance to a number of antimicrobial agents such as beta-lactam antibiotics, macrolides, quinolones, and vancomycin is becoming a major worldwide health problem (Cohen, M. L., Trends Microbiol., 2:422-425 (1994)). The most significant problem in clinical practice is the increase in the isolation of methicillin-resistant Staphylococcus aureus (MRSA) strains. In the United States, by the early 1990s MRSA was detected in 20-40% of all S. aureus hospital isolates reported to the National Nosocomial Infections Surveillance (NNIS) System and is also a major problem in long-term care facilities. In addition to resistance to beta-lactam antibiotics, multiply resistant MRSA are also resistant to macrolides, tetracyclines, aminoglycosides, and fluoroquinolones. At present, the only effective treatment for multiply resistant MRSA infections is vancomycin. However, the minimum inhibitory concentration (MIC) for vancomycin against some MRSA isolates has been increasing recently, leading to a situation where standard doses of vancomycin may not be effective for severe infections (Major Unmet Needs in Bacterial Infection Therapy. Infectious Disease, A Pharmacor Service, August, 1992.).

Consequently, much research has been done to study the mutual effect of simultaneously administered antibiotics, exerted on each other and on various pathogenic microorganisms. The studies performed by investigators show that the effect of simultaneously administered antibiotics is either synergism or antagonism. In the case of synergism, the antibiotic combination exhibits a marked increase in activity over that which could be predicted as the result of a purely additive effect of the two or more drugs in combination. Both quantitative and qualitative synergistic effects have been observed.

The treatment of infections due to multiple-antibiotic-resistant organisms presents a challenge which a number of clinicians have in the past sought to meet through the utilization of synergistic antibiotic combinations. The use of synergistic antibiotic combinations allows for the treatment of those more difficult infections at lower dosage levels than otherwise possible, thereby lowering the probability of toxicity complications, the time for treatment, and, potentially, the cost of therapy.

The combination of amoxicillin and potassium clavulanate for the treatment of sinusitis has been used by physicians. Seggev et al., Arch Otolaryngol Head Neck Surg, 124:921 (1998), compares the safety and efficacy of a combination of amoxicillin and clavulanate potassium given orally every 12 hours with that given every 8 hours for the treatment of patients with acute bacterial maxillary sinusitis. The study shows that amoxicillin and clavulanate given every 12 hours is as effective and as safe as administration every 8 hours for the treatment of acute bacterial maxillary sinusitis. However, Seggev et al. does not teach aerosolized delivery of a combination of antibiotics to patients with sinusitis.

Cefuroxime and gentamicin, either individually or in combination with another agent, have been used to treat patients with sinusitis (Gurses et al. J Antimicrob Chemother, 38:547 (1996); Boner et al., Int J Clin Pharmacol Ther Toxicol, 22:511 (1984); Koltai et al., Laryngoscope, 95:34 (1985)). Gurses et al. (1996) reports oral administration of cefuroxime to children between the ages of 5-14 suffering from acute sinusitis. Boner et al. (1984) discloses intramuscular administration of a combination of cefuroxime and N-acetyl-cysteine for the treatment of maxillary sinusistis in children. Koltai et al. (1985) describes the combination of Caldwell-Luc operation and postoperative intranasal instillation of gentamicin for the treatment of patients with chronic maxillary sinusitis. However, aerosolized delivery of a combination of cefuroxime and gentamicin for the treatment of sinusitis has not been reported.

### SUMMARY OF THE INVENTION

The present invention relates to pharmaceutical compositions that include one or more active ingredients such as an anti-infective agent, an anti-inflammatory agent, a mucolytic agent, an antihistamine, an antileukotriene, a decongestant, an anticholinergic agent, antifungal agent, and a combination of these classes of agents. Anti-infective agents contemplated by the present invention include, but are not limited to antibiotics, anti-virals, non-antibiotic antimicrobials, and antiseptics Anti-inflammatory agents contemplated by the present invention include but are not limited to steroidal and non-steroidal anti-inflammatory agents, and mast cell stabilizers. Antifungal agents contemplated by the present invention include but are not limited to amphotericin and azole antifingals, such as itraconazole, miconazole, and fluconazole. Combinations of antibiotics are also contemplated by the present invention.

Such compositions preferably are formulated as a liquid (solution, suspension, emulsion, etc.) or a powder, that can be mixed with diluent to produce a liquid, in a unit dose or multi-dose vial for aerosol administration to the nasal sinuses. It is contemplated that such formulations are packaged in association with labels or inserts or other forms of directions for their use in the treatment of sinusitis.

In a preferred embodiment, the surface tension of the solution or suspension is below about 70 dynes/cm, in order to yield an aerosol having a preferred mass median aerodynamic diameter within the range of about 1.0 to 5.0 microns. The use of such an aerosolized spray has minimal systemic side effects. It is preferable to have the maximum number of particles over about 5.0 microns to be less than about 20%.

Surface tension of a given formulation may be adjusted by adding a surfactant in addition to the active ingredients in order to bring it into the preferred range. More preferably, the surface tension is below about 55 dynes/cm, even more preferably, the surface tension is below about 50 dynes/cm, and most preferably, the surface tension is below about 45 dynes/cm. Even lower surface tensions are contemplated by the present invention. In one embodiment, the preferred range of surface tension is between about 10 to 40 dynes/cm. In another embodiment, the preferred range is between 20 to 40 dynes/cm. Most preferably, the surface tension is between about 30-40 dynes/cm.

Generally, it is contemplated that formulations according to the present invention will preferably have a pH in the range of about 3.0 to 8.5; an osmotic pressure of the solution or suspension between about 150 mOsm/kg to 880 mOsm/kg; and a NaCl equivalency to the solution or suspension is preferably between about 0.2% NaCl to 3.0% NaCl.

Preferred anti-infective agents include penicillins, cephalosporins, macrolides, ketolides, sulfonamides, quinolones, aminoglycosides, beta lactam antibiotics, and linezolid. Preferred non-antibiotic antimicrobials include taurolidine. Preferred steroidal anti-inflammatory agents include glucocorticoids. Preferred nonsteroidal anti-inflammatory agents include diclofenac. Preferred mast cell stabilizers include cromolyn and nedcromil sodium. Preferred mucolytic agents are acetylcysteine and domase alpha. Preferred decongestants are phenylephrine, naphazoline, oxymetazoline, tetrahydrozoline and xylometoazoline. Preferred antileukotrienes include montelukast. Preferred antihistamines include loratidine. Preferred antibiotic combinations include cefuroxime and gentamicin. Preferred antiseptics include iodine. Preferred anticholinergics include ipratropium, atropine, and scopolamine. Preferred antifungals include amphotericin B, itraconazole, fluconazole, and miconazole.

Preferred combinations of agents include, but are not limited to cefoperazone, oxymetazoline, and a decongestant; and ipratropium bromide and betamethasone.

In a preferred embodiment of the invention, a kit is described that provides the various equipment and attachments useful in administering the formulations of the present invention by using the disclosed nebulizer devices.

The present invention also contemplates methods of using the disclosed pharmaceutical compositions to treat mammals suspected or diagnosed to have sinusitis. In a preferred embodiment, the mammal is a human.

Preferred administration protocols also are described.

### BRIEF DESCRIPTION OF THE DRAWINGS

FIG. 1 discloses the preferred equipment for aerosolized delivery of pharmaceutical solutions or suspensions. This nebulizer, manufactured by Pari Respiratory Equipment, Inc., for the inventors, produces the desired particle size for effective administration of the solutions or suspensions in this invention to the sinuses. To use this nebulizer preferably medication is placed in the nebulizer at A. The nebulizer is then connected to a compressor or other source at B with tubing supplied. When the airflow is turned on the patient places the nosepiece C under their nostrils and breathes normally until the medication solution or suspension in the nebulizer begins to sputter and no mist comes out at C.

### DETAILED DESCRIPTION OF THE INVENTION

I. General Description

The present invention involves the topical delivery of medications to the nasal cavity and sinuses by aerosolizing aqueous solutions or suspensions of these medications. The present invention is based in part on the surprising finding that aerosolized anti-infective particles are surprisingly effective therapeutically when they have a mass median aerodynamic diameter (MMAD) of about 1.0 to 5.0 microns for deposition in the sinuses in a preferred size range. The present invention provides an apparatus for delivery of such optimally sized particles of anti-infectives or other active agents into the sinuses. The present invention is also based in part on the finding that the addition of a surfactant to formulations increases the deposition, retention, and penetration of anti-infectives or other active ingredients into the sinuses. The present invention provides guidance for therapy schedule and dosage as discussed in detail below.

As described in greater detail below, the pharmaceutical formulations will be aerosolized/atomized to form an aerosol cloud for nasal inhalation by the patient. This aerosol cloud will have liquid aerosol particles consisting of diluent and medication and having a MMAD of preferably between about 0.5 and 10 microns, more preferably between about 1.0 to 5.0 microns and most preferably between about 2.0 to 4.0 microns. Acceptable diluents, for example, may be water, saline solution, or a mixture of water and alcohol. It is also preferable to have the maximum number of particles over about 5.0 microns be less than about 20% of the total particles.

The size of the particles may be measured by laser diffraction, cascade impaction, or other methods known to one of ordinary skill in the art. Preferably, the aerosolized particles of the present invention are measured by laser diffraction.

A surprising discovery made by the inventors was that the surface tension of the solution or suspension prepared for inhalation needed to be adjusted to achieve optimal results. To achieve effective deposition of medication within the sinuses it is preferable to have the surface tension of the solution or suspension for aerosolization be adjusted with surfactants to less than about 70 dynes/cm, more preferably less than about 55 dynes/cm, even more preferably less than about 50 dynes/cm and most preferably between less than about 45 dynes/cm. Even lower surface tensions are contemplated. In one embodiment, the preferred surface tension is between about 10 to 40 dynes/cm. In another embodiment, the preferred surface tension is between about 20 to 40 dynes/cm. Most preferably, the surface tension is between about 30 to 40 dynes/cm.

Contemplated pharmaceutical compositions will include one or more active ingredients such as anti-infective agents, anti-inflammatory agents, mucolytic agents, antihistamines, antileukotrienes, decongestants, anticholinergics, antifungals, and combinations of these classes of agents. Anti-infective agents contemplated by the present invention include, but are not limited to antibiotics, anti-virals, non-antibiotic antimicrobials, and antiseptics. Anti-inflammatory agents contemplated by the present invention include, but are not limited to steroidal and non-steroidal anti-inflammatory agents, and mast cell inhibitors. Antifungal agents contemplated by the present invention include, but are not limited to amphotericin B, and azole antifungal. Examples of contemplated antibiotics include, but are not limited to cefuroxime, ciprofloxacin, tobramycin, cefoperazone, erythromycin, and gentamycin. Appropriate medications to be used in the methods according to the present invention are listed in Table 1. These medications may be administered for the treatment of sinusitis, particularly chronic sinusitis, by resolving infection, reducing inflammation or reducing congestion in the nasal cavity and sinuses.

These compositions ideally will be formulated into a liquid (solution, suspension, emulsion etc.) in a unit dose or multi-dose vial for aerosol administration to the nasal cavity and sinuses and being packaged with directions for its use in the treatment of sinusitis. The compositions include powder that can be mixed with a diluent to produce a liquid. Appropriate compositions for this purpose will be formulated by using surfactants, NaCl, or other chemical entities to adjust the liquid for administration to have the following properties:

surface tension preferably less than about 70 dynes/cm, more preferably less than about 55 dynes/cm, even more preferably less than about 50 dynes/cm, most preferably less than about 45 dynes/cm. Even lower surface tensions are contemplated by the present invention. In one embodiment, the preferred surface tension is between about 10 to 40 dynes/cm. In another embodiment, the preferred surface tension is between about 20 to 40 dynes/cm. Most preferably, the surface tension is between about 30 to 40 dynes/cm.

osmotic pressure between about 200 mOsm/kg to 880 mOsm/kg, more preferably between about 300 mOsm/kg to 700 mOsm/kg and most preferably between about 400 mOsm/kg to 550 mOsm/kg.

NaCl equivalency of the solution or suspension preferably between about 0.2% NaCl and 3.0% NaCl, more preferably between about 0.45% NaCl and 1.8% NaCl and most preferably between about 0.9% NaCl and 1.7% NaCl.

pH preferably between about 3.0 and 8.5, but may vary according to the properties of the medication used.

A. Surface Tension:

The present inventors have found that the surface tension and, to a lesser degree, particle size are critical factors in getting optimal deposition of the formulation in the nasal cavity and sinuses. For example, particles that are too large will deposit in the nasal cavity, but are unlikely to enter the sinuses. Lowering the surface tension increases an aerosolized particle's chance of deposition on surfaces that it contacts, i.e., the nasal cavities and sinus cavities. In contrast, liquids with surface tension in the range similar to that of water or higher will have more likelihood of being deposited in the lungs or being breathed back out into the atmosphere.

For purposes of preparing formulations according to the present invention, surface tension may be measured by using a ring tensiometer or the capillary rise measure method which consists of a capillary tube of known diameter placed into the liquid and a measurement of capillary rise taken to provide surface tension. Surface tension may also be measured by the spinning drop method, pendant drop method, bubble pressure method, drop volume method, and Wilhelmy plate method. Surface tension will then be adjusted using surfactants or agents capable of lowering surface tension to fall within a preferred range in dynes/cm.

B. Osmotic Pressure:

Optimal osmotic pressure helps to reduce damage to the epithelia cilia and mucosa of the sinuses. Although often not present in chronic sinusitis patients, epithelia cilia perform a useful function in the sinuses by moving mucosal fluid out of the sinuses.

For purposes of preparing formulations according to the present invention, osmotic pressure may be measured by using an Osmometer. If necessary, osmotic pressure may then be raised to fall within a preferred range by adding NaCl, dextrose, or other salts to the liquid.

C. Sodium Chloride Equivalency:

Optimal NaCl equivalency (tonicity) works to reduce swelling in the sinuses and nasal cavity by drawing water from the nasal and sinus epithelia, reducing swelling. NaCl equivalency below 0.9% (hypotonic) may cause swelling in the epithelia of the nasal cavity and sinuses. NaCl equivalency above 3.0% would raise the tonicity and osmotic pressure above desirable levels and may cause a burning sensation.

For purposes of preparing formulations according to the present invention, NaCl equivalency will closely follow osmotic pressure and can be measured using the methods described in section B above.

D. pH:

In general, the pH would be adjusted if a given medication is either more stable or more effective at a certain pH. American Hospital Formulary Service (AFHS) published yearly or the Hand Book of Injectable Drugs by Lawrence A. Trissel©, 1994 American Society of Hospital Pharmacists, Inc., which are herein incorporated by reference, provide information regarding the stability or effectiveness of a medication at certain pH.

For the purposes of preparing formulations according to the present invention the pH of the various liquids may need to be adjusted to achieve stability or increase effectiveness. A pH meter, where a probe is placed into the solution or suspension and the device gives the pH, will be used to measure pH, or pH paper will be used to estimate pH by placing liquid on the tape and then comparing to a predeveloped chart of pH colorations. When necessary, pH will then be adjusted to arrive at the most preferable range of pH needed for nasal aerosolization by adding buffering agents.

E. General Preparation of a Unit Dose and Production of Aerosol with Optimal Particle Diameter:

After determining the medications to be used in the formulation, each ingredient is weighed/measured out individually, added together, mixed with diluent, for example, sterile water, and filtered with a coarse filter and then a fine filter (5 micron, 2 micron, 1 micron, 0.45 micron, or 0.22 micron). The preparation is then tested to ensure that it is within the parameters established for surface tension, osmolarity, pH, and sodium chloride equivalency. This is done by using the appropriate equipment for each test as noted in Sections A to D above. To prepare a unit dose, the ingredients of such formulations generally will be dissolved in a solvent such as water or saline solution, in a volume between about 0.5 and 6.0 mls, more preferably between about 2 and 4 mls and most preferably between about 2.5 and 3.5 mls.

F. Surfactants:

The surface tension of a fluid is the tendency of the fluid to “stick” to itself when there is a surface between the liquid and the vapor phase (known as an interface). A good example is a drop of water falling in air. The drop assumes a spherical shape due to surface tension forces, which minimize its surface given the volume. Molecules at the surface of a liquid exert strong attractive forces on other molecules within their vicinity. The resultant force acting perpendicular to a line of unit length in the surface is known as surface tension, usually measured in Dynes/Centimeter.

Surfactants can be used as dispersing agents, solubilizing agents, and spreading agents. Some examples of surfactants are: PEG 400, sodium lauryl sulfate, spans (20-40-60 etc), tweens (polysorbates, 20-40-60 etc), tyloxapol, propylene glycol, and Benzalkonium chloride. Contemplated surfactants include any compound or agent that lowers the surface tension of a composition.

The purpose of using surfactants in the preferred formulations of the present invention is to adjust the surface tension of the aerosolized particles so that the maximum amount of medication is deposited within the sinus cavities. If the surface tension is reduced too much, the majority of the particles will deposit in the nasal cavity, conversely if the surface tension is too high the particles go directly to the lungs without depositing in the nasal sinuses.

The HLB (hydrophile-lipophile-balance) is used to describe the characteristics of a surfactant. The system consists of an arbitrary scale to which HLB values are experimentally determined and assigned. If the HLB value is low, the number of hydrophilic groups on the surfactant is small, which means it is more lipophilic (oil soluble).

Surfactants can act as a solubilizing agent by forming micelles. For example, a surfactant with a high HLB would be used to increase the solubility of an oil in an aqueous medium. The lipophilic portion of the surfactant would entrap the oil in the lipophilic (interior) portion of the micelle. The hydrophilic portion of the surfactant surrounding the oil globule would, in turn, be exposed to the aqueous phase.

An HLB value of 10 or higher means that the agent is primarily hydrophilic, while an HLB value of less than 10 means it would be lipophilic. For example, spans have HLB values ranging from 1.8 to 8.6, which is indicative of oil soluble for oil dispersible molecules. Consequently, the oil phase will predominate and a water/oil emulsion will be formed. Tweens have HLB values that range from 9.6 to 16.7, which is characteristic of water-soluble or water dispersible molecules. Therefore, the water phase will predominate and oil/water emulsions will be formed.

Emulsifying agents are surfactants that reduce the interfacial tension between oil and water, thereby minimizing the surface energy through the formation of globules. Wetting agents, on the other hand, aid in attaining intimate contact between solid particles and liquids.

Detergents are also surfactants that reduce the surface tension of a liquid to wet or spread over a solid surface. When a detergent is used, small particles in a liquid will be emulsified and foaming may occur.

One effect of adding surfactants to the formulations is smaller particle size. Effective particle sizes as low as 1 micron are contemplated. There are many ways to measure particle size. The particle size may be measured by using laser diffraction Laser diffraction is the most accurate way for measuring wet aerosols (droplets of liquids). Cascade impaction is a common method for measuring dry aerosols (solids in aerosolized powder). In cascade impaction, water is evaporated from the particles in the measuring process. As a result, the values are smaller than laser diffraction. Thus, the preferred method for measuring the size of particles in aerosols as contemplated by the present invention is by laser diffraction.

The present invention also contemplates the use of any compound or agent that lowers the surface tension of a liquid.

The preferred compound that acts like a surfactant, lowering the surface tension of the composition, is Pineapple Artificial Flavorings (Meridian Pharmaceuticals, Inc., Catalog No. FLA-218). This compound not only covers the smell and taste of some antibiotics but also has excellent surfactant properties. Additionally, it is less drying and irritating than other surfactants.

G. Pathogens Known to Produce Acute and Chronic Sinus Infections:

A retrospective review of sinus cultures obtained over a 4-year period from a consecutive series of patients who underwent endoscopic sinus surgery (ESS) was conducted by Niel Bhattacharyya M.D. et al., Archives of Otolaryngology—Head and Neck Surgery Vol. 125 No. 10, October 1999. A wide range of bacteria may be present in the infected post-ESS sinus cavity, with a considerable population of gram-negative organisms, including Pseudomonas species. Fungal infections of the sinuses have a nonspecific clinical presentation, are refractory to standard medical treatment and may produce expansion and erosion of the sinus wall. Various factors have been implicated in the development of fungal sinusitis: anatomical factors in the osteomeatal complex, tissular hypoxia, traumatic factors, massive exposure to fungal spores, allergy and immunosuppression.

The most common bacterial organisms found are the following: Alpha Hemolytic Streptococci, Beta Hemolytic Streptococci, Branhamella catarrhalis, Diptheroids, Haemophilis influenzae (beta-lactamase positive and negative), Moraxella species, Pseudomonas aeruginosa, Pseudomonas maltophilia, Serratia marcescens, Staphylococcus aureus, and Streptococcus pneumonia.

The most common fungal organisms found are the following: Aspergillis, Mucor and Candida Albicans, Fusarium, Curvularia, Cryptococcus, Coccidioides, and Histoplasma.

The optimum treatment modality is for the physician to obtain a bacterial/fungal culture from the sinus cavities via endoscopy, with a suction devise, or a swab. The culture is sent to a laboratory where it is tested for minimum inhibitory concentration for several antibiotics and then the correct antibiotic can be chosen based on the sensitivities provided by the laboratory. Current therapy by most Otolaryngologists is to determine the best antibiotic by using their clinical experience in treating sinus infections. This is called empiric therapy.

The anti-fungal therapy is done similarly in that it can also be cultured and sent to the lab for identification allowing the most effective agent to be prescribed, or empiric therapy is performed by the physician.

The kill rate is determined by the susceptibility of the organism to the antibiotic or antifungals. The kill is determined/measured by a repeat culture and sensitivity test showing no bacterial or fungal growth (as appropriate). If an effective anti-infective is used the infection usually resolves in a period of 10 days to three weeks.

H. Anti-leukotrienes

Inflammation plays an important role in the development of nasal polyps. Leukotrienes B4, C4, D4, and E4 are potent chemical mediators important in allergic inflammation. Leukotriene receptor antagonists (anti-leukotrienes) are a new class of drugs which target and block the action of these mediators.

Examples of leukotriene receptor antagonists include, but are not limited to, zafirlukast, montelukast, pranlukast, iralukast, and pobilukast.

It is contemplated that because of their effect, these medications applied topically according to the present invention will reduce inflammation in the nasal cavity and thereby help prevent the development of and also shrink existing polyps.

I. Antihistamines

Antihistamines are used for the relief of manifestations of immediate-type hypersensitivity reactions. Antihistamine effects include inhibition of respiratory, vascular and GI smooth muscle constriction; decreased capillary permeability, which reduces the wheal, flare, and itch response; and decreased histamine-activated exocrine secretions (e.g. salivary, lachrymal). Antihistamines with strong anticholinergic (atropine like) properties also can potentiate the drying effect by suppressing cholinergically innervated exocrine glands.

Examples of antihistamines include, but are not limited to, ethanolamines such as diphenyhydramine, carbinoxamine, clemastine, phenytoloxamine, doxylamine, dimenhydrinate, and bromodiphenhydramine hydrochloride; ethylenediamines such as tripelennamine, pyrilamine, antazoline, and methapyriline; alkylamines such as pheniramine, chlorpheniramine, brompheniramine, dexchlorpheniramine, dimethindene, and triprolidine; phenothiazines such as promethazine, trimeprazine, propiomazine and methdilazine; piperazines such as hydroxyzine (hydrochloride and pamoate), cyclizine, chlorcyclizine, buclizine and meclizine; and miscellaneous antihistamines such as cyproheptidine, azatadine, diphenylpyraline, ketotifen, terfenadine, fexofenadine, astemizole, and phenindamine.

Providing antihistamines according to the present invention will help those patients needing relief of manifestations of immediate-type hypersensitivity reactions.

J. Antiseptics

Examples of antiseptics include, but are not limited to, iodine, chlorhexidine acetate, sodium hypochlorite, and calcium hydroxide. Iodine or a salt thereof such as povidone iodine, potassium iodine, and sodium iodine, is the preferred iodine.

Iodine preparations are used externally for their broad microbicidal spectrum against bacteria, fungi, viruses, spores, protozoa and yeasts.

Providing potassium iodide according to the present invention is believed to be a more effective way to provide the medication to a greater area within the sinus cavity resulting in relief of bacteria, fungi, viruses, spores, protozoa and yeasts infections.

K. Antibiotic Combinations

Providing a combination of anti-bacterial agents according to the present invention consisting of two or more antibiotics with differing spectra of activity allows a physician to cover a wider spectrum of the offending bacterial organisms found in chronic sinusitis. Examples of some appropriate antibiotics are shown in Table 1.

L. Steroidal Anti-Inflammatories

Examples of steroidal anti-inflammatories include, but are not limited to, betamethasone, triamcinolone, dexamethasone, prednisone, mometasone, fluticasone, beclomethasone, flunisolide, and budesonide.

These drugs have potent glucocorticoid and weak mineralocorticoid activity. The mechanisms responsible for the anti-inflammatory action of corticosteroids on the nasal mucosa are unknown. However, glucocorticoids have a wide range of inhibitory activities against multiple cell types (e.g., histamine, eicosanoids, leukotrienes, cytokines) involved in allergic and nonallergic/irritant-mediated inflammation. These agents, when administered topically in recommended doses, exert direct local anti-inflammatory effects, including hypothalamic-pituitary-adrenal (HPA) function suppression.

Providing steroidal anti-inflammatories according to the present invention is believed to be a more effective way to provide the medication to a greater area within the sinus cavity resulting in a decrease of the release of mediating factors and reduce inflammation.

M. Non-Steroidal Anti-Inflammatories

Examples of nonsteroidal anti-inflammatory agents include, but are not limited to, fenoprofen, flurbiprofen, ibuprofen, ketoprofen, naproxen, oxaprozin, diclofenac, etodolac, indomethacin, ketorolac, nabumetone, sulindac tolmetin meclofenamate, mefenamic acid, piroxicam and suprofen.

Nonsteroidal anti-inflammatory drugs have analgesic and antipyretic activities. Exact mode of action is not known. Major mechanism is believed to be inhibition of cyclooxygenase activity and prostaglandin syntheses. Other mechanisms may exist as well, such as inhibition of lipoxygenase, leukotriene synthesis, lysosomal enzyme release, neutrophil aggregation and various cell membrane functions.

Providing nonsteroidal anti-inflammatory agents according to the present invention will help those patients needing relief from nasal inflammation.

N. Decongestants

Examples of decongestants include, but are not limited to phenylpropanolamine, pseudoephedrine, phenylephrine, epinephrine, ephedrine, desoxyephedrine, naphazoline, oxymetazoline, tetrahydrozoline, xylometazoline and propylhexedrine.

Decongestants stimulate alpha adrenergic receptors of vascular smooth muscle (vasoconstriction, pressor effects, nasal decongestion), although some retain beta adrenergic properties (e.g., ephedrine, pseudoephedrine). Other alpha effects include contraction of the G.I. and urinary sphincters, mydriasis and decreased pancreatic beta cell secretion. The alpha adrenergic effects cause intense vasoconstriction when applied directly to mucous membranes; systemically, the products have similar muted effects and decongestion occurs without drastic changes in blood pressure, vascular redistribution or cardiac stimulation. Constriction in the mucous membranes results in their shrinkage; this promotes drainage, thus improving ventilation and the stuffy feeling.

Decongestant sympathomimetic amines are administered directly to swollen membranes (e.g., via spray, drops, nebulizer) or systemically via the oral route. They are used in acute conditions such as hay fever, allergic rhinitis, vasomotor rhinitis, sinusitis and the common cold to relieve membrane congestion.

Providing decongestants according to the present invention will help those patients needing relief of mucous membrane congestion.

O. Mucolytics

Examples of mucolytics include, but are not limited to acetylcysteine, and dornase alpha.

Acetylcysteine: The viscosity of mucus secretions depends on the concentration of mucoprotein in the secretory fluid, the presence of disulfide bonds between these macromolecules, and to a lesser extent, the presence of DNA. The mucolytic action of acetylcysteine is related to the sulfhydryl group in the molecule, which acts directly to split disulfide linkages between mucoprotein molecular complexes, resulting in depolymerization and a decrease in mucus viscosity. The action is unaffected by the presence of DNA. The mucolytic activity of acetylcysteine increases with increasing pH. Significant mucolysis occurs between pH 7 and 9.

Dornase alpha: A highly purified solution of rhDNase (recombinant human deoxyribonuclease I), an enzyme that selectively cleaves DNA. In vitro, domase hydrolyzes the DNA in sputum and reduces sputum viscoelasticity.

Providing these medications according to the present invention will help to reduce mucus viscosity and viscoelasticity providing better drainage and evacuation of mucus build up within the sinuses.

P. Anticholinergics

Examples of anticholinergics include, but are not limited to ipratropium, atropine, and scopolamine.

Anticholinergics prevent the increases in intracellular concentrations of cyclic guanosine monophosphate, which are caused by interaction of acetylcholine with the muscarinic receptor of some smooth muscles. Specifically ipratropium has been shown to be affective in patients with allergic or nonallergic perennial rhinitis, where studies showed there was a statistically significant decrease in the severity and duration of rhinorrhea.

Providing anticholinergics according to the present invention will help reduce the amount of perennial rhinitis the patient suffers.

Q. Non-Antibiotic Antimicrobials

Examples of non-antibiotic antimicrobials include, but are not limited to taurolidine.

Non-antibiotic antimicrobials exhibit their activity by disrupting cell wall synthesis, diminishing bacterial adherence to mucosal walls, and neutralizing endotoxins. Specifically taurolidine, which is broken down into the amino acid taurine, not only has bactericidal activity but also has been shown to have antilipopolysaccharide activity and primes polymorphonuclear leukocytes luminal diameters for enhanced antimicrobial activity.

Providing these medications according to the present invention will help by allowing the use of a non-antibiotic to treat bacterial and fungal infections, which disrupts cell wall synthesis of bacteria, diminishes adherence to mucosal walls of bacteria and fungi, as well as neutralize endotoxins released by bacteria such as Staphylococcus aureus.

R. Mast Cell Stabilizers

Examples of mast cell stabilizers include, but are not limited to cromolyn and nedocromil sodium.

Mast cell stabilizers are antiasthmatic and antiallergic. Mast cell stabilizers inhibit the degranulation of sensitized and nonsensitized mast cells, which occurs after exposure to specific antigens. The drug inhibits the release of histamine and SRS-A (the slow reacting substance of anaphylaxis, a leukotriene) from the mast cell.

Providing mast cell inhibitors according to the present invention will help those patients needing relief of rhinorrhea, nasal congestion, sneezing and postnasal drip.

II. Specific Embodiments

A. Pharmaceutical Compositions and Formulations

Preferred anti-infective agents include penicillins, cephalosporins, macrolides, ketolides, sulfonamides, quinolones, aminoglycosides, beta lactam antibiotics, and linezolid. Preferred anti-inflammatory agents include glucocorticoids, disodium cromoglycate, and nedcromil sodium. Preferred mucolytic agents are acetylcysteine and dornase alpha. Preferred decongestants are phenylephrine, naphazoline, oxymetazoline, tetrahydrozoline, and xylometoazoline. Preferred antileukotrienes include montelukast. Preferred antihistamines include loratidine. Preferred anticholinergics include ipratropium, atropine, and scopolamine. Preferred antiseptic includes iodine. Preferred antifungals include amphotericin B and azole antifungals. Preferred non-antibiotic antimicrobial includes taurolidine. Preferred non-steroidal anti-inflammatory agent includes diclofenac. These agents may be found in the American Hospital Formulary Service published by American Society of Hospital Phannacists, Inc., which is incorporated herein by reference.

As an example of a contemplated formulation, cefuroxime is formulated in dosages of 285 mg in 3 ml sterile water for injection per dose, to produce an antibiotic for aerosol administration. This formulation may be compounded under a laminar flow hood by performing the following steps: 1) weigh out sufficient cefuroxime to provide 21 doses of 285 mg each (5985 mg), with 5% overage to account for that lost in compounding; 2) QS ad (add up to) to 63 ml with sterile water, with 5% overfill for loss in compounding; and 3) add 0.1 ml of polysorbate 20 per 100 ml liquid. The final compounded liquid mixture is filtered using a 0.22 micron filter before placing in a unit of use (unit dose) container.

The surface tension of the formulation is measured using a ring tensiometer. Alternatively, the surface tension may be determined by measuring the capillary rise of the formulation. The preferable range of surface tension for the formulation of this present invention is 10 to 70 dynes/cm. The formulation may be adjusted with a surfactant if necessary using, for example, polysorbate 20, to obtain the preferred surface tension.

Using a pH meter, the formulation is tested for the desirable pH, preferably in the range of about 3.0 to 8.5. The pH is adjusted with appropriate acids, bases and appropriate buffers as needed according to conventional compounding practices.

Preferably the formulation will also be evaluated using E tables from sources known to practitioners skilled in the pharmaceutical arts, such as Remington: The Science and Practice of Pharmacy or other suitable pharmaceutical text to calculate its sodium chloride equivalence to ensure that it is in the preferred range of 0.2% to 1.5%. Similarly, the osmolarity is checked to ensure that it falls within the preferred range of about 300 to 880 mOsm/kg. If osmolarity falls outside of this range, the polysorbate 20 component may be decreased until the preferred conditions are met.

As a second example, ciprofloxacin is formulated in dosages of 90 mg unit dose in 3 ml of sterile water for injection per dose.

This formulation may be compounded under a laminar flow hood by performing the following steps: 1) weigh out a sufficient quantity of ciprofloxacin powder to prepare 28 doses (2520 mg) with 5% overage to account for loss during compounding; 2) QS ad to 74 ml sterile water for injection (add 5% overage for loss in compounding); and 3) add 0.25 ml polysorbate 20 for every 100 ml of liquid. The final compounded liquid mixture is filtered using a 0.22 micron filter before placing in a unit of use (unit dose) container.

The formulation is tested as described above and adjustments made to bring surface tension, pH, sodium chloride equivalence, and osmolarity within preferred ranges or to preferred levels.

As a third example, amphotericin B is formulated in 10 mg unit doses along with hydrocortisone sodium succinate in 50 mg unit doses in 3 ml sterile water to provide an antifungal agent together with an anti-inflammatory agent.

This formulation may be compounded under a laminar flow hood by performing the following steps: 1) weigh out sufficient powder of amphotericin B to make 28 doses (280 mg) of 10 mg each allowing 5% overage for loss in compounding; 2) weigh out sufficient powder of hydrocortisone sodium succinate to make 28 doses (1400 mg) of 50 mg each allowing 5% overage for loss of compounding; 3) combine powders; and 4) QS ad sterile water for injection to 84 ml plus 5% for loss in compounding. The final compounded liquid mixture is filtered using a 0.45 micron or 1 micron filter before placing in a unit of use (unit dose) container. A filter with a larger pore is necessary for filtering amphotericin.

The formulation is tested as described above and adjustments made to bring surface tension, pH, sodium chloride equivalence, and osmolarity within preferred ranges or to preferred levels.

As a fourth example, ofloxacin is formulated in 90 mg unit doses along with acetylcysteine in 100 mg unit doses in 3 ml of sterile water to provide an antibiotic together with a mucolytic agent.

This formulation is compounded under a laminar flow hood by performing the following steps: 1) weigh out sufficient powder of ofloxacin to make 28 doses (2520 mg) of 90 mg each allowing 5% overage for loss in compounding; 2) weigh out sufficient powder of acetylcysteine to make 28 doses (2800 mg) of 100 mg each allowing 5% overage for loss in compounding; and 3) combine the powders and QS ad to 84 ml with sterile water for injection allowing 5% overage for loss during compounding. The final compounded liquid mixture is filtered using a 0.22 micron filter before placing in a unit of use (unit dose) container.

The formulation is tested as described above and adjustments made to bring surface tension, pH, sodium chloride equivalence, and osmolarity within preferred ranges or to preferred levels.

As a fifth example, tobramycin is formulated in 100 mg unit doses in 2.5 ml of saline solution to provide an alternative antibiotic formulation. The formulation is compounded under a laminar flow hood by performing the following steps: 1) weigh out sufficient tobramycin powder to provide 42 doses of 100 mg per dose (4200 mg), allowing for 5% overage due to losses during compounding; 2) QS ad with 105 ml of sterile water for injection, allowing for 5% overage due to losses during compounding; and 3) add 0.15 ml polysorbate 20 to adjust surface tension. The final compounded liquid mixture is filtered using a 0.22 micron filter before placing in a unit of use (unit dose) container.

The formulation is tested as described above and adjustments made to bring surface tension, pH, sodium chloride equivalence, and osmolarity within preferred ranges or to preferred levels.

As a sixth example, cefoperazone and oxymetazoline are formulated in 3 ml of sterile water for injection to provide an antibiotic formulated with a decongestant. This formulation is prepared under a laminar flow hood by following these steps: 1) weigh out sufficient powder of cefoperazone to make 28 doses of 600 mg each (16.8 g) allowing 5% overage for compounding loss; 2) weigh out sufficient powder of oxymetazonline to make 28 doses of 0.5 mg each (14 mg) allowing 5% overage for compounding loss; 3) combine the powders together; 4) QS ad with sterile water to 84 ml allowing 5% overage for compounding loss; 5) add benzalkonium chloride 0.02% (0.02 gm/100ml of liquid). The final compounded liquid mixture is filtered using a 0.22 micron filter before placing in a unit of use (unit dose) container.

The formulation is tested as described above and adjustments made to bring surface tension, pH, sodium chloride equivalence, and osmolarity within preferred ranges or to preferred levels.

As a seventh example, montelukast is formulated in dosages of 3.5 mg in 3 ml of sterile water for injection per dose.

This formulation may be compounded under a laminar flow hood by performing the following steps: 1) crush five tablets of montelukast with a mortar and pestle; 2) solubilize the powder with sterile water for injection; 3) gross filter the solution or suspension with filter paper; 4) sterile filter the resultant mixture with a 0.22 micron filter; and 5) Qs ad to 42 ml with sterile water for injection with 5% overage for loss in compounding.

The surface tension of the formulation is measured using a ring tensiometer. The preferable range is 10 to 70 dynes/cm. The formulation may be adjusted with a surfactant, for example, polysorbate 20. Using a pH meter, the formulation is tested for the desirable pH, preferably in the range of about 3.0 to 8.5. The pH is adjusted with appropriate acids, bases and appropriate buffers as needed according to conventional compounding practices. In addition the formulation will also be evaluated using E tables from sources known to practitioners skilled in the pharmaceutical arts, such as Remington: Science and Practice of Pharmacy or other suitable pharmaceutical text to calculate its sodium chloride equivalence to ensure that it is in the preferred range of 0.9% to 3.0%. Similarly, the Osmolarity is checked to ensure that it falls within the preferred range of about 300 to 880 mOsm/kg. If osmolarity falls outside of this range, the polysorbate 20 component may be decreased until the preferred conditions are met.

As an eighth example, loratidine is formulated in dosages of 2 mg in 3 ml of sterile water for injection per dose.

This formulation may be compounded under a laminar flow hood by performing the following steps: 1) crush three tablets (lOmg each) in a mortar and pestle; 2) add 0.5 ml of 0.125% polysorbate 20 to the powder and triturate until the powder is wet; 3) add 30 ml of sterile water for injection and mix well; 4) gross filter with filter paper; 5) sterile filter with a 0.22 micron filter; and 6) QS ad with sterile water for injection to a final volume of 45 ml (may allow 5% overage for compounding loss).

The surface tension of the formulation is measured using a ring tensiometer. The preferable range is 10 to 70 dynes/cm. The formulation may be adjusted with a surfactant if necessary using, for example, polysorbate 20. Using a pH meter, the formulation is tested for the desirable pH, preferably in the range of about 3.0 to 8.5. The pH is adjusted with appropriate acids, bases and appropriate buffers as needed according to conventional compounding practices. In addition the formulation will also be evaluated using E tables from sources known to practitioners skilled in the pharmaceutical arts, such as Remington: Science and Practice of Pharmacy or other suitable pharmaceutical text to calculate its sodium chloride equivalence to ensure that it is in the preferred range of 0.9% to 3.0%. Similarly, the osmolarity is checked to ensure that it falls within the preferred range of about 300 to 880 mOsm/kg. If osmolarity falls outside of this range, the polysorbate 20 component may be decreased until the preferred conditions are met.

As a ninth example, a combination antibiotic preparation consisting of gentamicin 95 mg and cefuroxime 285 mg in unit dose in 4.5ml sterile water for injection. In the following, gentamicin and cefuroxime are stated as the activity of the drug.

This formulation may be compounded under a laminar flow hood by performing the following steps: 1) weigh out sufficient quantity of gentamicin powder to prepare 42 doses (3990 mg) with 5% overage to account for loss during compounding; 2) weigh out sufficient quantity of cefuroxime powder to prepare 42 doses (11,970 mg) with 5% overage to account for loss during compounding; 3) mix the powders and QS ad to 252 ml with sterile water for injection; 4) test physical properties as above and adjust as necessary; and 5) sterile filter with 0.22 micron filter.

As a tenth example, potassium iodide 2% is formulated in dosages of 60 mg unit dose in 3 ml sterile water for injection per dose.

This formulation may be compounded under a laminar flow hood by performing the following steps: 1) weigh out a sufficient quantity of potassium iodide to prepare 42 doses (2520 mg) with 5% overage to account for loss during compounding; 2) QS ad to 126 ml with sterile water for injection with 5% overage for loss during compounding; 3) test liquid as above and ensure the pH is between 7.5 and 4.5; and 4) sterile filter the final liquid with 0.22 micron filter.

As an eleventh example ipratropium bromide and betamethasone are formulated in 3 ml of sterile water/normal saline for injection to provide an anticholinergic agent formulated with an anti-inflammatory agent.

This formulation is prepared under a laminar flow hood by following these steps:

1) weigh out sufficient powder of ipratropium bromide to provide the number of doses needed at 0.075 mg per dose with 5% overage for compounding losses; 2) using one half of the total volume of liquid to be made, dissolve ipratropium bromide in normal saline (use 5% overage for compounding losses); 3) weigh out sufficient powder of betamethasone phosphate to provide the number of doses needed at 0.4 mg per dose betamethasone activity also allowing for 5% overage for compounding losses; the activity is noted on the manufacturer container label or can be gotten from the supplier; 4) using one half of the total volume of liquid to be made, dissolve betamethasone in sterile water with 5% overage for compounding losses; and 5) combine the two solutions or suspensions. The final compounded liquid mixture is filtered using a 0.22 micron filter before dispensing in 3 ml aliquots to the unit of use (unit dose) containers. This formulation is tested as described above and adjustments made to bring surface tension, pH, sodium chloride equivalence, and osmolarity within preferred ranges or to preferred levels.

As a twelfth example taurolidine can be formulated into 3 ml of sterile water/normal saline for injection to provide a non-antibiotic antimicrobial for nebulization.

This formulation is prepared under a laminar flow hood by following these steps:

1) weigh out sufficient powder of taurolidine to provide 80 mg per dose with 5% overage for compounding losses; 2) dissolve the powder using a suitable diluent (sterile water, normal saline, povidone) allowing 5% overage for compounding; and 3) divide the resultant solution into 3ml aliquots to the unit of use containers. The formulation is tested as described earlier. Adjustments are made to bring surface tension, pH, sodium chloride equivalence, and osmolarity within preferred ranges or to preferred levels.

As a thirteenth example, diclofenac is formulated in dosages of 1.0 mg in 3 ml of sterile water per dose.

This formulation may be compounded under a laminar flow hood by performing the following steps: 1) remove the enteric coating from a 25 mg tablet; 2) crush the tablet using a mortar and pestle; 3) solubilize the powder with sterile water; 4) gross filter the solution with filter paper; 5) sterile filter the resultant mixture with a 0.22 micron filter; and 6) QS ad to 75ml with sterile water with 5% overage for loss in compounding. The solution is then tested as described above. Adjustments are made to bring surface tension, pH, sodium chloride equivalence, and osmolarity within preferred ranges or to preferred levels.

As a fourteenth example, cromolyn is formulated in 5 mg unit doses along with acetylcysteine 100 mg unit doses in 3 ml of sterile water to provide a mast cell stabilizer with a mucolytic.

The formulation is compounded under a laminar flow hood be performing the following steps: 1) weigh out sufficient quantity of cromolyn powder to make the number of doses required, adding 5% for compounding losses; 2) weigh out sufficient powder of acetylcysteine to make the number of doses required, adding 5% for compounding losses; and 3) combine the powders and QS ad with sterile water to sufficient volume to make the number of 3 ml doses asked for in the prescription. The final solution is filtered using a 0.22-micron filter before placing in a unit of use (unit dose) container.

The formulation is tested as described above. Adjustments are made to bring surface tension, pH, sodium chloride equivalence, and osmolarity within preferred ranges or to preferred levels.

B. Determination of the Course of Treatment

In general, the course of treatment for any given patient will be determined by his or her physician. Thus, if the organisms found in a patient's sinuses are cultured by known techniques and their sensitivities are determined, the most appropriate antibiotic and/or antifungal will be ordered. However, if no cultures and sensitivities are done, then the patient also may be treated empirically with the antibiotic or antifungal chosen by the physician using his or her experience based on what bacteria or fungus is suspected. If the anatomical structures inside the nasal passageways are swollen or inflamed due to allergy or flu symptoms, an anti-inflammatory agent and/or a decongestant agent also may be administered if the patient is not otherwise using nasal sprays or oral medication separately.

Example of a Patient Treatment Scenario Involving Sinus Infections:

1. Patient contracts what he/she feels is a sinus infection and goes to his/her otolaryngologist for diagnosis. After determining the diagnosis of sinusitis, a culture is obtained endoscopically and sent to the laboratory.

2. The laboratory determines the bacteria/fungus sensitivities by drug and reports its findings to the physician.

3. The physician faxes the report to the pharmacy along with a prescription for the antibiotic most appropriate for the infection. The formulation is prepared as described above and dispensed in 2.5 ml containers. Generally, the container will be labeled: “Store in Refrigerator.”

4. The pharmacist will call patient and discuss the treatment and any pertinent data necessary to enhance the treatment outcome.

Example of a Treatment Scenario Involving a Patient with Polyps:

1. The patient presents to the otolaryngologist with symptomatic nasal obstruction caused by nonatopic rhinosinusitis or allergic rhinosinusitis.

2. The physician orders a CT scan of the sinus region and evaluates the patient's condition.

3. If the diagnosis is nasal polyposis, the physician can treat non invasively and with little to no side effects using nebulized corticosteroids. (The therapy in current use consists of surgery and/or high dose of corticosteroids either intravenously or orally. Surgery is invasive, and corticosteroids may induce many unwanted side effects.)

4. The physician would fax a prescription order to the pharmacy asking for the corticosteroid to be nebulized, in an amount most appropriate for the treatment of this patient.

5. The formulation is prepared, labeled and packaged for the patient under the supervision of a licensed pharmacist in 3 ml unit of use containers.

C. Contemplated and Preferred Treatment Regimens:

The preferred treatment is the antibiotic (adjusted for the proper surface tension, pH, sodium chloride equivalence, and osmolarity) that most effectively kills the bacteria or fungus as determined by culture and sensitivity, administered once to three times per day for a duration of 5 to 10 minutes per each treatment (See Table 1).

The total number of days needed to rid the infection preferably is determined by reculturing until no growth is noted. However, when the physician does not do culturing, the conventional standard of practice is two weeks of therapy until patient generally would be expected to have become asymptomatic plus an additional 7 days of therapy.

D. Monitoring Efficacy:

The typical otolaryngologist when treating chronic sinusitis prescribes antibiotics until the patient is symptom free by physical exam plus an additional seven days. The problem that occurs with respect to sinus infections is that, if the infection is not completely resolved, the patient will have a recurrence the next time his/her immune system is challenged, i.e., the next upper respiratory infection that results in obstruction of the osteomeatal complex, impairs mucociliory clearance and causes over production of secretions. Thus, the preferred method of determining resolution of the infection is to reculture the sinuses endoscopically and have the laboratory report come back negative, i.e., reporting no growth of pathogenic microorganisms. The present inventors have discovered that aerosolization should lead to less resistance exhibited by bacteria due to the fewer times they are exposed to the antibiotic, and such exposure occurs at lower dosages and for shorter periods of time of aerosolized administration (typically 1-3 weeks) as compared to oral (typically 3 weeks to several months) and intravenous treatment (typically 3-6 weeks).

E. Equipment for Aerosolized Delivery of Pharmaceutical Composition:

Equipment for aerosolized delivery of pharmaceutical compositions is well known to the skilled artisan. O'Riordan et al., Journal of Aerosol Medicine, 20(1): 13-23 (1997), reports the delivery of aerosolized tobramycin by a jet nebulizer and an ultrasonic nebulizer. U.S. Pat. No. 5,508,269, issued Apr. 16, 1996, compares the characteristics of three different nebulizers: the Ultraneb 99 (DeVilbiss) ultrasonic nebulizer, the Medicaid Sidestream jet nebulizer, and the Pari LC jet nebulizer.

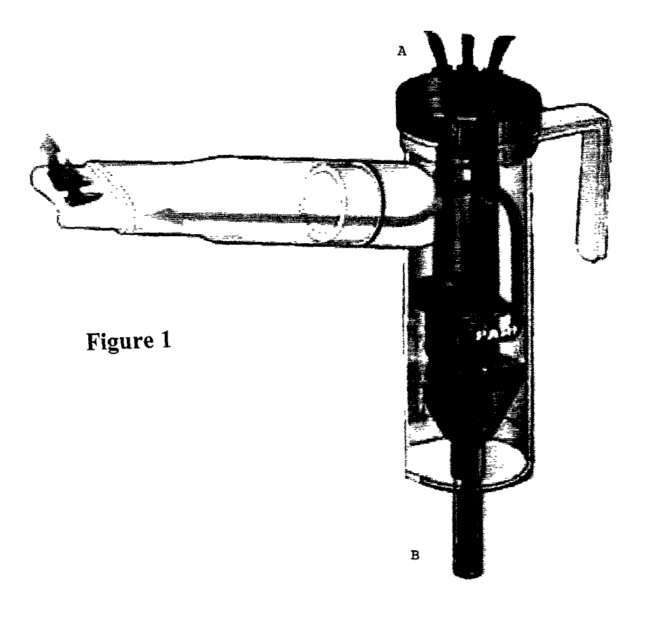

The preferred equipment for aerosolized delivery of pharmaceutical liquid is depicted in FIG. 1. This nebulizer manufactured by Pari Respiratory Equipment, Inc., for the inventors produces the desired particle size for effective administration of the liquid in this invention to the sinuses. To use this nebulizer, preferably 0.5 ml to 8 ml of liquid medication, more preferably 2 ml to 4 ml and most preferably 2.5 ml to 3.5 ml of liquid medication is placed in the nebulizer at A. The nebulizer is then connected to a compressor or other source to provide 4 liter/minute airflow at B with tubing supplied. When the airflow is turned on the patient places the nosepiece C under his/her nostrils and breathes normally until the liquid medication in the nebulizer begins to sputter and no mist comes out at C. This will usually take 8 to 12 minutes.

In light of the foregoing general discussion, the specific examples presented below are illustrative only and are not intended to limit the scope of the invention. Other generic and specific configurations will be apparent to those persons skilled in the art.

### EXAMPLES

### Example 1

Patient A

A female in her forties had been suffering from sinusitis for most of her adult life. These sinusitis episodes seemed to be triggered by allergies. She historically had three-four (3-4) episodes of sinusitis each year, which were treated with oral antibiotics for four-eight (4-8) weeks per episode. These oral antibiotic regimens produced yeast infections, which were treated with Diflucan® (fluconazole). Relief from the headaches, malaise, facial pressure and pain, yellow-green nasal discharge, coughing and fever took up to six weeks and were treated with narcotic and non narcotic analgesics, decongestants, decongestant nasal sprays, cough suppressants, and nasal rinses. Her allergies were treated with antihistamines and anti-inflammatory agents.

In an effort to reduce the duration of her sinusitis episodes, a nose drop of tobramycin 80 mg/ml was administered. This treatment did not seem to work. The medication was irritating; and in order to administer the drops and try to get them into the sinus cavity, the patient had to hold her head back. This caused intolerable pain resulting in the discontinuation of the therapy. A nose drop of Bactroban® was tried. It was not efficacious; it was very viscous. The administration of this drop produced similar pain on administration, and this therapy was also discontinued.

In order to eliminate the pain caused by holding her head back when administering nose drops, a nose drop of tobramycin was administered after the patient had been on oral antibiotics for a period of time. This did not seem to work. The drop did not seem to penetrate into the sinus cavities.

Thereafter, a preparation of tobramycin 80 mg/ml was administered using 3 ml in a Pari LC Star® nebulizer cup with adult mask attached and a Pari Proneb® compressor. The medication was nebulized three (3) times daily. After four days of therapy, the patient experienced a “dumping” of green, purulent nasal discharge. The therapy was continued for a total of seven (7) days. It seemed at this point that the sinus infection had been eliminated, but a relapse was experienced within a month. Another seven (7) day regimen of nebulized tobramycin was given to the patient. Again the sinus infection seemed to be eliminated, but it reoccurred within two (2) months.

A preparation of cefuroxime 285 mg in 2.5 ml sterile water for injection was administered three (3) times daily using a Pari LC Star® nebulizer cup with adult mask attached and a Pari Proneb® compressor. The time of nebulization was extensive and the medication did not seem to be completely nebulized. After one day of therapy, a Pari Turbo® compressor was substituted for the Pari Proneb® compressor. The patient experienced a “dumping” of green, purulent nasal discharge after (3) days of therapy. The therapy was continued for a total of seven (7) days, again she contracted a yeast infection and was given Diflucan®.

After the seven (7) days of treatment with nebulized cefuroxime using the Pari Turbo® compressor and the Pari LC Star® nebulizer cup with mask, the patient remained free of sinus infections for nine (9) months. She continued to experience problems with her allergies, and while in the past these allergies triggered sinus infections, this time no such infection recurred.

### Example 2

Patient B

A male in his forties had been experiencing sinus infections off and on during his adult life. He was treated with cefuroxime 285 mg in 2.5 ml of sterile water for injection three (3) times daily using a Pari LC Star® nebulizer cup with adult mask attached and a Pari Turbo® compressor. The patient experienced a “dumping” of green, purulent nasal discharge after eight (8) treatments. The therapy was continued for a total of seven (7) days. No other antibiotics were given. This patient remained free from sinus infections for six (6) months.

### Example 3

Patient C

A female aged mid-fifty had been suffering from sinusitis off and on for most of her adult life. These sinusitis episodes seemed to be triggered by allergies. The patient took antihistamines and decongestants when allergies triggered headaches and/or a clear nasal discharge. Historically, she would have one or more sinus infections a year requiring twenty or more days of oral antibiotics.

She was treated with cefuroxime 285 mg in 2.5 ml of sterile water for injection three (3) times daily using a Pari LC Star® nebulizer cup with adult mask attached and a Pari Turbo® compressor. The patient experienced a “dumping” of green, purulent nasal discharge after eight (8) treatments. The therapy was continued for a total of seven (7) days. No other antibiotics were given. This patient remained free from sinus infections for six (6) months.

It should be understood that the foregoing discussion and examples merely present a detailed description of certain preferred embodiments. It therefore should be apparent to those of ordinary skill in the art that various modifications and equivalents can be made without departing from the spirit and scope of the invention. All journal articles, other references, patents and patent applications that are identified in this patent application are incorporated by reference in their entirety.

TABLE 1
Agents and Dosages

| | | | | | More | | Most | | Most |
|---|---|---|---|---|---|---|---|---|---|
| | Brand | | Preferable | | Preferable | | Preferable | | Preferable |
| Generic Name | Name | Class | Range | | Range | | Range | | Dose |
| Acetylcysteine | Mucomist | Mucolytics | 125-500 | mg | 150-450 | mg | 200-400 | mg | 300 mg Q12H |
| | Mucosil | | | | | | | | |
| Amikacin | Amikin | Aminoglycoside | 50-500 | mg | 75-300 | mg | 100-200 | mg | 166 mg |
| | | | | | | | | | Q8-12H |
| Amphotericin B | Fungizone | Antifungal | 2.5-45 | mg | 4-30 | mg | 7.5-15 | mg | 10 mg Q12H |
| Atropine | | Anticolinergic | 10-700 | mcg | 25-400 | mcg | 75-300 | mcg | 200 mcg |
| | | | | | | | | | Q12H |
| Azelastine | Astelin | Antihistamine | 137- | | 204-822 | mcg | 382-616 | mcg | 411 mcg |
| | | | 1096 | mcg | | | | | Q12H |
| Azithromycin | Zithromax | Macrolide | 50-400 | mg | 75-300 | mg | 150-200 | mg | 167 mg Q12H |
| Aztreonam | Azactam | Monobactam | 250-1000 | mg | 300-900 | mg | 475-750 | mg | 450 mg Q8H |
| Beclamethasone | Vanceril | Steroidal Anti- | 0.1-4 | mg | 0.2-3 | mg | 0.2-2 | mg | 0.8 mg Q12H |
| | Beclovent | inflammatory | | | | | | | |
| Betamethasone | Celestone | Steroidal Anti- | 0.1-4 | mg | 0.2-3 | mg | 0.2-2 | mg | 0.8 mg Q12H |
| | | inflammatory | | | | | | | |
| Cefazolin | Ancef, | Cephlasporin | 250-1000 | mg | 300-900 | mg | 575-700 | mg | 650 mg Q8H |
| | Kefzol | (Gen I) | | | | | | | |
| Cefepime | Maxipime | Cephlasporin | 125-1000 | mg | 200-900 | mg | 575-700 | mg | 650 mg Q12H |
| | | (Gen IV) | | | | | | | |
| Cefonicid | Moniacid | Cephlasporin | 250-1000 | mg | 300-900 | mg | 575-700 | mg | 600 mg Q24H |
| | | (Gen II) | | | | | | | |
| Cefoperazone | Cefobid | Cephlasporin | 250-1000 | mg | 300-900 | mg | 575-700 | mg | 600 mg Q12H |
| | | (Gen III) | | | | | | | |
| Cefotaxime | Claforan | Cephlasporin | 250-1000 | mg | 300-900 | mg | 575-700 | mg | 600 mg |
| | | (Gen III) | | | | | | | Q8-12H |
| Cefotetan | Cefotan | Cephlasporin | 250-1000 | mg | 300-900 | mg | 575-700 | mg | 600 mg |
| | | (Cephamycin) | | | | | | | Q8-12H |
| Cefoxitin | Mefoxin | Cephlasporin | 250-1000 | mg | 300-900 | mg | 575-700 | mg | 600 mg Q12H |
| | | (Cephamycin) | | | | | | | |
| Ceftazidime | Fortaz, | Cephlasporin | 250-1000 | mg | 300-900 | mg | 475-750 | mg | 550 mg Q12H |
| | Ceptaz | (Gen III) | | | | | | | |
| Ceftizoxime | Cefizox | Cephlasporin | 250-1000 | mg | 300-900 | mg | 575-700 | mg | 600 mg |
| | | (Gen III) | | | | | | | Q8-12H |
| Ceftriaxone | Rocephin | Cephlasporin | 250-1000 | mg | 300-900 | mg | 575-700 | mg | 650 mg Q12H |
| | | (Gen III) | | | | | | | |
| Cefuroxime | Ceftin | Cephlasporin | 100-600 | mg | 200-520 | mg | 250-400 | mg | 285 mg Q8H |
| | | (Gen II) | | | | | | | |
| Cephapirin | Cefadyl | Cephlasporin | 250-1000 | mg | 300-900 | mg | 575-700 | mg | 650 mg Q12H |
| | | (Gen I) | | | | | | | |
| Ciprofloxacin | Cipro | Quinolone | 25-200 | mg | 50-175 | mg | 75-110 | mg | 90 mg Q12H |
| Clindamycin | Cleocin | Lincosamide | 50-600 | mg | 75-500 | mg | 125-300 | mg | 225 mg Q12H |
| Cromolyn | Intal/ | Mast cell | 5-100 | mg | 7.5-75 | mg | 10-50 | mg | 20 mg Q12H |
| Sodium | Nasalcrom | stabilizer | | | | | | | |
| Dexamethasone | Decadron | Steroidal Anti- | 0.1-4 | mg | 0.2-3 | mg | 0.2-2 | mg | 0.8 mg Q12H |
| | | inflammatory | | | | | | | |
| Dornase alpha | Pulmozyme | Mucolytic | 0.5-5 | mg | 1-4 | mg | 2-3 | mg | 1.5 mg Q12H |
| Doxycycline | Vibramycin | Tetracycline | 10-100 | mg | 15-80 | mg | 25-65 | mg | 27 mg Q12H |
| Erythromycin | Erythrocin | Macrolide | 50-600 | mg | 60-350 | mg | 100-300 | mg | 150 mg Q8H |
| Lactobionate | | | | | | | | | |
| Fluconazole | Diflucan | Antifungal | 12.5-150 | mg | 20-70 | mg | 25-50 | mg | 30 mg Q12H |
| Flunisolide | Aerobid | Steroidal Anti- | 0.1-4 | mg | 0.2-3 | mg | 0.2-2 | mg | 0.8 mg Q12H |
| | Nasalide | inflammatory | | | | | | | |
| Flurbiprofen | Ocufen | Nonsteroidal | 0.01-2 | mg | 0.05-1 | mg | 0.1-0.5 | mg | 0.15 mg |
| | | Anti- | | | | | | | Q12H |
| | | inflammatory | | | | | | | |
| Fluticasone | Flonase | Steroidal Anti- | 10-700 | mcg | 25-400 | mcg | 75-300 | mcg | 200 mcg |
| | | inflammatory | | | | | | | Q24H |
| Gentamycin | Garamycin | Aminoglycoside | 10-200 | mg | 30-150 | mg | 80-120 | mg | 95 mg |
| | | | | | | | | | Q8-12H |
| Ibuprofen | Motrin | Nonsteroidal | 25-400 | mg | 30-300 | mg | 50-150 | mg | 100 mg Q12H |
| | | Anti- | | | | | | | |
| | | inflammatory | | | | | | | |
| Ipratropium | Atrovent | Anticholinergic | 10-700 | mcg | 25-400 | mcg | 75-300 | mcg | 200 mcg |
| | | | | | | | | | Q12H |
| Itraconazole | Sporanox | Antifungal | 12.5-150 | mg | 20-70 | mg | 25-50 | mg | 30 mg Q12H |
| Ketorolac | Acular | Nonsteroidal | 0.05-4 | mg | 0.1-2 | mg | 0.3-1 | mg | 0.5 mg Q12H |
| | | Anti- | | | | | | | |
| | | inflammatory | | | | | | | |
| Levofloxacin | Levaquin | Quinolone | 40-200 | mg | 50-150 | mg | 60-80 | mg | 70 mg Q12H |
| Linezolid | Zyvox | Miscellaneous | 50-600 | mg | 75-450 | mg | 100-300 | mg | 200 mg Q12H |
| | | anti-bacterial | | | | | | | |
| Loratidine | Claritin | Antihistamine | 0.5-10 | mg | 1-7.5 | mg | 1-5 | mg | 2 mg Q12H |
| Meropenem | Merrin | Carbapenem | 200-750 | mg | 250-700 | mg | 300-500 | mg | 33 mg Q8H |
| Mezlocillin | Mezlin | Penicillin | 300-1500 | mg | 375-1000 | mg | 750-950 | mg | 833 mg Q6H |
| Miconazole | Monistat | Antifungal | 12.5-300 | mg | 30-200 | mg | 50-100 | mg | 60 mg Q12H |
| Montelukast | Singulair | Antileukotriene | 0.5-15 | mg | 2-25 | mg | 3-15 | mg | 10 mg Q12H |
| Mupirocin | Bactroban | Antibacterial | 1-25 | mg | 1.5-20 | mg | 2-15 | mg | 10 mg Q6-8H |
| Nafcillin | Unipen | Penicillin | 250-1000 | mg | 300-900 | mg | 575-700 | mg | 600 mg Q8H |
| Nedocromil | Tilade | Mast cell | 1-25 | mg | 3-15 | mg | 5-12 | mg | 7 mg Q12H |
| | | stabilizer | | | | | | | |
| Ofloxacin | Floxin | Quinolone | 25-200 | mg | 50-175 | mg | 75-110 | mg | 90 mg Q12H |
| Oxacillin | Prostaphlin | Penicillin | 250-1000 | mg | 300-900 | mg | 575-700 | mg | 600 mg Q8H |
| Oxymetazoline | Afrin | Decongestant | 0.05-0.5 | mg | 0.075-0.4 | mg | 0.1-0.3 | mg | 0.2 mg Q12H |
| Phenylepherine | Neo- | Decongestant | 5-50 | mg | 10-35 | mg | 15-20 | mg | 10 mg Q12H |
| | Synephine | | | | | | | | |
| Piperacillin | Pipracil | Penicillin | 100-1000 | mg | 125-750 | mg | 250-600 | mg | 460 mg Q6H |
| Potassium | — | Antiseptic | 30-200 | mg | 40-150 | mg | 50-80 | mg | 60 mg q12h |
| Iodide | | | | | | | | | |
| Rifampin | Rifadin | Miscellaneous | 500-5000 | mg | 1000-4000 | mg | 1500-3500 | mg | 2250 mg |
| | | | | | | | | | Q12H |
| Taurolin | Taurolidine | Non antibiotic | 5-200 | mg | 20-150 | mg | 40-120 | mg | 80 mg Q12H |
| | | antimicrobial | | | | | | | |
| Tetrahy- | Tizine | Decongestant | 0.05-0.5 | mg | 0.06-0.4 | mg | 0.1-0.3 | mg | 0.15 mg |
| drozolidine | | | | | | | | | Q12H |
| Ticarcillin + | Timentin | Penicillin | 500-5000 | mg | 1000-4000 | mg | 1500-3500 | mg | 2250 mg |
| Clavulanate | | | | | | | | | Q6-8H |
| Tobramycin | Nebcin | Aminoglycoside | 10-200 | mg | 30-150 | mg | 80-120 | mg | 95 mg |
| | | | | | | | | | Q8-12H |
| Triamcinalone | Asthmacor | Steroidal Anti- | 0.05-3 | mg | 0.2-2.5 | mg | 0.5-2 | mg | 0.6 mg Q12H |
| | Aristocort | inflammatory | | | | | | | |
| Vancomycin | Vancocin | Antibiotic- | 50-400 | mg | 75-325 | mg | 125-250 | mg | 166 mg |
| | | miscellaneous | | | | | | | Q6-8H |
| Xylometazoline | Otrivin | Decongestant | 0.05-0.4 | mg | 0.075-0.3 | mg | 0.1-0.2 | mg | 0.125 mg |
| | | | | | | | | | Q12H |
| Zafirlukast | Accolate | Antileukotriene | 2-60 | mg | 4-50 | mg | 6-30 | mg | 20 mg Q12H |

1. A pharmaceutical composition comprising one or more active ingredients selected from the group consisting of an anti-infective agent, anti-inflammatory agent, mucolytic agent, antihistamine, anticholinergic agent, an antileukotriene, a decongestant, and a combination of these classes of agents, said composition being formulated as a liquid in a unit dose for aerosol administration to the nasal sinuses and being packaged with directions for its use in the treatment of sinusitis, and wherein the surface tension of the liquid is less than about 70 dynes/cm.

2. The composition of claim 1, wherein the composition has a pH in the range of about 3.0 to 8.5.

3. The composition of claim 1, wherein the anti-infective agent is selected from the group consisting of an antibiotic, an anti-viral, a non-antibiotic antimicrobial, and an antiseptic.

4. The composition of claim 1, wherein the anti-inflammatory agent is selected from the group consisting of steroidal anti-inflammatory agents, non-steroidal anti-inflammatory agents, and mast cell inhibitors.

5. The composition of claim 1, wherein the composition further comprises a compound that lowers the surface tension of the liquid to less than about 70 dynes/cm in addition to the one or more active ingredients.

6. The composition of claim 5, wherein the compound is a surfactant.

7. The composition of claim 1, further comprising a decongestant.

8. The pharmaceutical composition of claim 1, which when administered as an aerosolized spray has minimal systemic side effects.

9. The pharmaceutical composition of claim 1, wherein said active ingredient is an anti-infective agent and the sinusitis is caused by a pathogen selected from the group consisting of Alpha Hemolytic Streptococci, Beta Hemolytic Streptococci, Branhamella Catarrhalis, Diptheroids, Haemophilis influenzae (beta-lactamase positive and negative), Moraxella species, Pseudomonas aeruginosa, Pseudomonas maltophilia, Serratia marcescens, Staphylococcus aureus, Streptococcus pneumonia, Aspergillosis, Mucor and Candida Albicans, Fusarium, Curvularia, cryptococcus, coccidioides, and histoplasma.

10. The pharmaceutical composition of claim 9, wherein said anti-infective agent is selected from the group consisting of penicillins, cephalosporins, macrolides, ketolides, sulfonamides, quinolones, aminoglycosides, beta lactam antibiotics, and linezolid.

11. The pharmaceutical composition of claim 6, wherein said surfactant is a polysorbate.

12. A kit containing a pharmaceutical composition of claim 1, and a nebulizer cup with a nasal adapter for delivering the composition in aerosolized form to the nasal sinuses.

13. The combination of a nebulizer device and the kit of claim 12.

14. An aerosol comprising the pharmaceutical composition of claim 1, wherein at least about 85% of the aerosolized particles have a mass median aerodynamic diameter within the range of about 1.0 to 5.0 microns.

15. An aerosol according to claim 14, wherein said aerosol is effective to kill at least about 90% of pathogens causing sinusitis in a patient within about 21 days following an every 8 hr (TID), every 12 hr (BID), or every 24 hr (AD) administration protocol.

16. A method of treating a mammal suspected or diagnosed as having chronic sinusitis comprising the step of administering to the patient the pharmaceutical composition of any one of claims 1 or 2, by aerosolization using a nebulizer which delivers aerosol particles of between about 1 to 5 microns in average diameter.

17. The method of claim 16, wherein the mammal is a human.

18. The method of claim 16, wherein the nebulizer is a PARI nebulizer with a nasal adapter.

19. The method of claim 16, wherein the nebulizer is connected to a PARI SinuNEB compressor.

20. The method of claim 16, wherein the nebulizer delivers a majority of aerosolized particles in the size range of about 2.0 to 4.0 microns in diameter.

21. The method claim 16, wherein the pharmaceutical composition is administered to the patient 1-3 times a day for a total of 7-28 days.

22. The pharmaceutical composition of claim 10, wherein the polysorbate is selected from the group consisting of polysorbate 20 to polysorbate 85.

23. The composition of claim 3, wherein the antibiotic is selected from the group consisting of cefuroxime, ciprofloxacin, amphotericin B, ofloxacin, and tobramycin.

24. The composition of claim 3, wherein the non-antibiotic antimicrobial is taurolidine.

25. The composition of claim 3, wherein the antiseptic is iodine or a salt thereof.

26. The composition of claim 1, wherein the antileukotriene is montelukast.

27. The composition of claim 1, wherein the antihistamine is loratidine.

28. The composition of claim 1, wherein the active ingredients are a combination of antibiotics comprising gentamicin and cefuroxime or cefoperazone and oxymetazoline.

29. The pharmaceutical composition of claim 1, wherein the surface tension is less than about 50 dynes/cm.

30. The pharmaceutical composition of claim 1, wherein the surface tension is between about 10 to 40 dynes/cm.

31. A pharmaceutical composition comprising one or more antifungal agents, said composition being formulated as a liquid in a unit dose for aerosol administration to the nasal sinuses and being packaged with directions for its use in the treatment of sinusitis, and wherein the surface tension of the liquid is less than about 45 dynes/cm.

32. The pharmaceutical composition of claim 31, wherein the surface tension is between about 10 to 40 dynes/cm.

33. The pharmaceutical composition of claim 31, wherein the antifungal agents are selected from the group consisting of amphotericin B, and azole antifungals.

34. An aerosol comprising the pharmaceutical composition of claim 31, wherein at least about 85% of the aerosolized particles have a mass median aerodynamic diameter within the range of about 1.0 to 5.0 microns.

35. A method of treating a mammal suspected or diagnosed as having chronic sinusitis comprising the step of administering to a patient the pharmaceutical composition of claim 31, by aerosolization using a nebulizer which delivers aerosol particles of between about 1 to 5 microns in average diameter.

36. A method of treating nasal or sinus polyps comprising administering to a patient the composition of claim 1.

37. The method of claim 36, wherein the composition comprises a steroidal anti-inflammatory and a diluent.
